SEQUENCE LISTING (21 sequences)

SEQ ID NO: 1 - protein, 293 aa, unidentified
  REGION 1..293: note = Description of sequence: Enzyme: Wild type Leaf-branch compost cutinase (LCC)
MDGVLWRVRTAALMAALLALAAWALVWASPSVEAQSNPYQRGPNPTRSALTADGPFSVATYTVSRLSVSGFGGGVIYYPTGTSLTFGGIAMSPGYTADASSLAWLGRRLASHGFVVLVINTNSRFDYPDSRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSTTPKVYVELDNASHFAPNSNNAAISVYTISWMKLWVDNDTRYRQFLCNVNDPALSDFRTNNRHCQ

SEQ ID NO: 2 - protein, 293 aa, synthetic construct
  REGION 1..293: note = Description of sequence: Enzyme: ICCM (LCCF243I/D238C/S283C/N246M)
MDGVLWRVRTAALMAALLALAAWALVWASPSVEAQSNPYQRGPNPTRSALTADGPFSVATYTVSRLSVSGFGGGVIYYPTGTSLTFGGIAMSPGYTADASSLAWLGRRLASHGFVVLVINTNSRFDYPDSRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSTTPKVYVELCNASHIAPMSNNAAISVYTISWMKLWVDNDTRYRQFLCNVNDPALCDFRTNNRHCQ

SEQ ID NO: 3 - protein, 293 aa, synthetic construct
  REGION 1..293: note = Description of sequence: Enzyme: LCCD238K
MDGVLWRVRTAALMAALLALAAWALVWASPSVEAQSNPYQRGPNPTRSALTADGPFSVATYTVSRLSVSGFGGGVIYYPTGTSLTFGGIAMSPGYTADASSLAWLGRRLASHGFVVLVINTNSRFDYPDSRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSTTPKVYVELKNASHFAPNSNNAAISVYTISWMKLWVDNDTRYRQFLCNVNDPALSDFRTNNRHCQ

SEQ ID NO: 4 - protein, 293 aa, synthetic construct
  REGION 1..293: note = Description of sequence: Enzyme: LCCF243I/D238K/N246M
MDGVLWRVRTAALMAALLALAAWALVWASPSVEAQSNPYQRGPNPTRSALTADGPFSVATYTVSRLSVSGFGGGVIYYPTGTSLTFGGIAMSPGYTADASSLAWLGRRLASHGFVVLVINTNSRFDYPDSRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSTTPKVYVELKNASHIAPMSNNAAISVYTISWMKLWVDNDTRYRQFLCNVNDPALSDFRTNNRHCQ

SEQ ID NO: 5 - protein, 259 aa, synthetic construct
  REGION 1..259: note = Description of sequence: Enzyme: ICCMD129E
MSNPYQRGPNPTRSALTADGPFSVATYTVSRLSVSGFGGGVIYYPTGTSLTFGGIAMSPGYTADASSLAWLGRRLASHGFVVLVINTNSRFDYPESRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSTTPKVYVELCNASHIAPMSNNAAISVYTISWMKLWVDNDTRYRQFLCNVNDPALCDFRTNNRHCQ

SEQ ID NO: 6 - protein, 259 aa, synthetic construct
  REGION 1..259: note = Description of sequence: Enzyme: LCCT229Q
MSNPYQRGPNPTRSALTADGPFSVATYTVSRLSVSGFGGGVIYYPTGTSLTFGGIAMSPGYTADASSLAWLGRRLASHGFVVLVINTNSRFDYPDSRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSQTPKVYVELDNASHFAPNSNNAAISVYTISWMKLWVDNDTRYRQFLCNVNDPALSDFRTNNRHCQ

SEQ ID NO: 7 - protein, 259 aa, synthetic construct
  REGION 1..259: note = Description of sequence: Enzyme: ICCMT229Q
MSNPYQRGPNPTRSALTADGPFSVATYTVSRLSVSGFGGGVIYYPTGTSLTFGGIAMSPGYTADASSLAWLGRRLASHGFVVLVINTNSRFDYPDSRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSQTPKVYVELCNASHIAPMSNNAAISVYTISWMKLWVDNDTRYRQFLCNVNDPALCDFRTNNRHCQ

SEQ ID NO: 8 - protein, 259 aa, synthetic construct
  REGION 1..259: note = Description of sequence: Enzyme: LCCD129E
MSNPYQRGPNPTRSALTADGPFSVATYTVSRLSVSGFGGGVIYYPTGTSLTFGGIAMSPGYTADASSLAWLGRRLASHGFVVLVINTNSRFDYPESRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSTTPKVYVELDNASHFAPNSNNAAISVYTISWMKLWVDNDTRYRQFLCNVNDPALSDFRTNNRHCQ

SEQ ID NO: 9 - protein, 259 aa, synthetic construct
  REGION 1..259: note = Description of sequence: Enzyme: LCCD129E/T229Q
MSNPYQRGPNPTRSALTADGPFSVATYTVSRLSVSGFGGGVIYYPTGTSLTFGGIAMSPGYTADASSLAWLGRRLASHGFVVLVINTNSRFDYPESRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSQTPKVYVELDNASHFAPNSNNAAISVYTISWMKLWVDNDTRYRQFLCNVNDPALSDFRTNNRHCQ

SEQ ID NO: 10 - protein, 259 aa, synthetic construct
  REGION 1..259: note = Description of sequence: Enzyme: LCCD129E/D238K
MSNPYQRGPNPTRSALTADGPFSVATYTVSRLSVSGFGGGVIYYPTGTSLTFGGIAMSPGYTADASSLAWLGRRLASHGFVVLVINTNSRFDYPESRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSTTPKVYVELKNASHFAPNSNNAAISVYTISWMKLWVDNDTRYRQFLCNVNDPALSDFRTNNRHCQ

SEQ ID NO: 11 - protein, 259 aa, synthetic construct
  REGION 1..259: note = Description of sequence: Enzyme: LCCT229Q/D238K
MSNPYQRGPNPTRSALTADGPFSVATYTVSRLSVSGFGGGVIYYPTGTSLTFGGIAMSPGYTADASSLAWLGRRLASHGFVVLVINTNSRFDYPDSRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSQTPKVYVELKNASHFAPNSNNAAISVYTISWMKLWVDNDTRYRQFLCNVNDPALSDFRTNNRHCQ

SEQ ID NO: 12 - protein, 259 aa, synthetic construct
  REGION 1..259: note = Description of sequence: Enzyme: ICCMD129E/T229Q
MSNPYQRGPNPTRSALTADGPFSVATYTVSRLSVSGFGGGVIYYPTGTSLTFGGIAMSPGYTADASSLAWLGRRLASHGFVVLVINTNSRFDYPESRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSQTPKVYVELCNASHIAPMSNNAAISVYTISWMKLWVDNDTRYRQFLCNVNDPALCDFRTNNRHCQ

SEQ ID NO: 13 - protein, 259 aa, synthetic construct
  REGION 1..259: note = Description of sequence: Enzyme: ICCMD129E/D238K/C283S
MSNPYQRGPNPTRSALTADGPFSVATYTVSRLSVSGFGGGVIYYPTGTSLTFGGIAMSPGYTADASSLAWLGRRLASHGFVVLVINTNSRFDYPESRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSTTPKVYVELKNASHIAPMSNNAAISVYTISWMKLWVDNDTRYRQFLCNVNDPALSDFRTNNRHCQ

SEQ ID NO: 14 - protein, 259 aa, synthetic construct
  REGION 1..259: note = Description of sequence: Enzyme: ICCMT229Q/D238K/C283S
MSNPYQRGPNPTRSALTADGPFSVATYTVSRLSVSGFGGGVIYYPTGTSLTFGGIAMSPGYTADASSLAWLGRRLASHGFVVLVINTNSRFDYPDSRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSQTPKVYVELKNASHIAPMSNNAAISVYTISWMKLWVDNDTRYRQFLCNVNDPALSDFRTNNRHCQ

SEQ ID NO: 15 - protein, 259 aa, synthetic construct
  REGION 1..259: note = Description of sequence: Enzyme: ICCMD129E/ T229Q/D238K /C283S
MSNPYQRGPNPTRSALTADGPFSVATYTVSRLSVSGFGGGVIYYPTGTSLTFGGIAMSPGYTADASSLAWLGRRLASHGFVVLVINTNSRFDYPESRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSQTPKVYVELKNASHIAPMSNNAAISVYTISWMKLWVDNDTRYRQFLCNVNDPALSDFRTNNRHCQ

SEQ ID NO: 16 - protein, 259 aa, synthetic construct
  REGION 1..259: note = Description of sequence: Enzyme: LCCD129E/ T229Q/D238K
MSNPYQRGPNPTRSALTADGPFSVATYTVSRLSVSGFGGGVIYYPTGTSLTFGGIAMSPGYTADASSLAWLGRRLASHGFVVLVINTNSRFDYPESRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSQTPKVYVELKNASHFAPNSNNAAISVYTISWMKLWVDNDTRYRQFLCNVNDPALSDFRTNNRHCQ

SEQ ID NO: 17 - protein, 308 aa, synthetic construct
  REGION 1..308: note = Description of sequence: Enzyme: Cut190
MRIRRQAGTGARASMARAIGVMTTALAVLVGAVGGVAGAEVSTAQDNPYERGPDPTEDSIEAIRGPFSVATERVSSFASGFGGGTIYYPRETDEGTFGAVAVAPGFTASQGSMSWYGERVASQGFIVFTIDTNTRLDQPGQRGRQLLAALDYLVERSDRKVRERLDPNRLAVMGHSMGGGGSLEATVMRPSLKASIPLTPWNLDKTWGQVQVPTFIIGAELDTIASVRTHAKPFYESLPSSLPKAYMELDGATHFAPNIPNTTIAKYVISWLKRFVDEDTRYSQFLCPNPTDRAIEEYRSTCPYLEHH

SEQ ID NO: 18 - protein, 308 aa, synthetic construct
  REGION 1..308: note = Description of sequence: Enzyme: Cut190D250K
MRIRRQAGTGARASMARAIGVMTTALAVLVGAVGGVAGAEVSTAQDNPYERGPDPTEDSIEAIRGPFSVATERVSSFASGFGGGTIYYPRETDEGTFGAVAVAPGFTASQGSMSWYGERVASQGFIVFTIDTNTRLDQPGQRGRQLLAALDYLVERSDRKVRERLDPNRLAVMGHSMGGGGSLEATVMRPSLKASIPLTPWNLDKTWGQVQVPTFIIGAELDTIASVRTHAKPFYESLPSSLPKAYMELKGATHFAPNIPNTTIAKYVISWLKRFVDEDTRYSQFLCPNPTDRAIEEYRSTCPYLEHH

SEQ ID NO: 19 - protein, 259 aa, synthetic construct
  REGION 1..259: note = Description of sequence: Enzyme: PHL7
MANPYERGPDPTESSIEAVRGPFAVAQTTVSRLQADGFGGGTIYYPTDTSQGTFGAVAISPGFTAGQESIAWLGPRIASQGFVVITIDTITRLDQPDSRGRQLQAALDHLRTNSVVRNRIDPNRMAVMGHSMGGGGALSAAANNTSLEAAIPLQGWHTRKNWSSVRTPTLVVGAQLDTIAPVSSHSEAFYNSLPSDLDKAYMELRGASHLVSNTPDTTTAKYSIAWLKRFVDDDLRYEQFLCPAPDDFAISEYRSTCPF

SEQ ID NO: 20 - protein, 259 aa, synthetic construct
  REGION 1..259: note = Description of sequence: Enzyme: PHL7R205K
MANPYERGPDPTESSIEAVRGPFAVAQTTVSRLQADGFGGGTIYYPTDTSQGTFGAVAISPGFTAGQESIAWLGPRIASQGFVVITIDTITRLDQPDSRGRQLQAALDHLRTNSVVRNRIDPNRMAVMGHSMGGGGALSAAANNTSLEAAIPLQGWHTRKNWSSVRTPTLVVGAQLDTIAPVSSHSEAFYNSLPSDLDKAYMELKGASHLVSNTPDTTTAKYSIAWLKRFVDDDLRYEQFLCPAPDDFAISEYRSTCPF

SEQ ID NO: 21 - protein, 259 aa, synthetic construct
  REGION 1..259: note = Description of sequence: Enzyme: ICCMD238K/C283S
MSNPYQRGPNPTRSALTADGPFSVATYTVSRLSVSGFGGGVIYYPTGTSLTFGGIAMSPGYTADASSLAWLGRRLASHGFVVLVINTNSRFDYPDSRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSTTPKVYVELKNASHIAPMSNNAAISVYTISWMKLWVDNDTRYRQFLCNVNDPALSDFRTNNRHCQ

(19) United States
(12) Patent Application Publication
(10) Pub. No.: US 2025/0179448 A1
(43) Pub. Date: Jun. 5, 2025

(54) LEAF-BRANCH COMPOST CUTINASE MUTANTS

(71) Applicant: Board of Regents, The University of Texas System, Austin, TX (US)

(72) Inventors: Hongyuan LU, Austin, TX (US); Daniel DIAZ, Austin, TX (US); Andrew ELLINGTON, Austin, TX (US); Hal ALPER, Austin, TX (US)

(73) Assignee: Board of Regents, The University of Texas System, Austin, TX (US)

(21) Appl. No.: 18/836,712

(22) PCT Filed: Feb. 7, 2023
(86) PCT No.: PCT/US2023/062092

§ 371 Date: Aug. 7, 2024

Related U.S. Application Data
(60) Provisional application No. 63/308,631, filed on Feb. 10, 2022.

(51) Int. Cl. C12N 9/18; C08J 11/10; C12N 1/20; C12N 15/78; C12N 15/80; C12R 1/40; C12R 1/645

(52) U.S. Cl. CPC C12N 9/18; C08J 11/105; C12N 1/20; C12N 15/78; C12N 15/80; C12R 2001/40; C12R 2001/645; C12Y 301/01074

(57) ABSTRACT

Leaf-branch compost cutinase (LCC) and other cutinase mutants with activity over ranges of temperature and pH are provided.

### CROSS-REFERENCE TO RELATED PATENT APPLICATIONS

The present application claims benefit of priority to U.S. Provisional Patent Application No. 63/308,631, filed Feb. 10, 2022, which is incorporated by reference for all purposes.

A Sequence Listing conforming to the rules of WIPO Standard ST.26 is hereby incorporated by reference. Said Sequence Listing has been filed as an electronic document via PatentCenter in ASCII format encoded as XML. The electronic document, created on Feb. 1, 2023, is entitled “093331-1363786-010710WO_ST26.xml”, and is 33,188 bytes in size.

### BACKGROUND OF THE INVENTION

Nowadays, plastics have become the most ubiquitous man-made materials in our daily life. Due to their excellent physical and chemical properties, they have been manufactured to a wide variety of products that are indispensable in many industries, such as packaging, clothing, automotive, electronics, household, agriculture, building and construction. The global production of plastics has reached 359 million tonnes in 2018, yet our current demand for plastics is still rapidly increasing.

Ironically, while most plastics are short-lived products that are designed for single use, they have a remarkably long lifespan attributed to their highly stable molecular structure. As a result of the ever-increased production and consumption of the recalcitrant plastic products, the accumulation of plastic waste has posed a growing threat to our ecosystems. Conventional plastic waste management technologies, such as combustion or pyrolysis, are often energy-intensive processes that could produce additional environmental pollutants. In contrast, the recently emerged enzymatic degradation of hydrolysable plastics can function under mild conditions and potentially recover plastics monomers from the depolymerization reaction. Thus, it has caught much attention from both academic and industrial communities. In particular, considerable progress has been made recently with enzymatic hydrolysis of PET, the fourth-most-produced plastic polymer.

Over the past decade, a number of enzymes were found to be capable of cleaving the ester bond between the PET monomers: terephthalate (TPA) and ethylene glycol (EG). Representatively, TfCut1 and TfCut2 from Thermobifida fusca KW3 (Furukawa, Makoto et al., 2019. “Efficient Degradation of Poly(Ethylene Terephthalate) with Thermobifida Fusca Cutinase Exhibiting Improved Catalytic Activity Generated Using Mutagenesis and Additive-Based Approaches.” Scientific Reports. https://doi.org/10.1038/s41598-019-52379-z; Then et al. 2016; 2015; Herrero Acero et al. 2011), Cut190 from Saccharomonospora viridis AHK190 (Oda, Masayuki et al., 2018. “Enzymatic Hydrolysis of PET: Functional Roles of Three Ca2+ Ions Bound to a Cutinase-like Enzyme, Cut190*, and Its Engineering for Improved Activity.” Applied Microbiology and Biotechnology. https://doi.org/10.1007/s00253-018-9374-x; Kawai, Fusako et al., 2014. “A Novel Ca2+-Activated, Thermostabilized Polyesterase Capable of Hydrolyzing Polyethylene Terephthalate from Saccharomonospora Viridis AHK190.” Applied Microbiology and Biotechnology. https://doi.org/10.1007/s00253-014-5860-y), and LC-cutinase derived from leaf compost metagenome (Tournier, V. et al., 2020. “An Engineered PET Depolymerase to Break down and Recycle Plastic Bottles.” Nature. https://doi.org/10.1038/s41586-020-2149-4; Shirke, Abhijit N et al., 2018. “Stabilizing Leaf and Branch Compost Cutinase (LCC) with Glycosylation: Mechanism and Effect on PET Hydrolysis.” Biochemistry. https://doi.org/10.1021/acs.biochem.7b01189) have been reported to have relatively high PET-degrading activity compared to other PET hydrolases. Recently, Yoshida et. al reported that a novel PET-assimilating bacterium Ideonella sakaiensis 201-F6 was successfully isolated from a PET bottle recycling site Yoshida, Shosuke et al., 2016. “A Bacterium That Degrades and Assimilates Poly(Ethylene Terephthalate).” Science 351 (6278): 1196 LP-1199. https://doi.org/10.1126/science.aad6359. This bacterium secreted a cutinase-like enzyme—IsPETase that exhibits the highest PET degradation activity among other PET-degrading enzymes identified so far at ambient temperature, indicating its promising environmental applicability. To develop a whole-cell biocatalyst system for environmental PET remediation, researchers have attempted to install IsPETase in several different chassis, including yeast (Pichia pastoris GS115, Yarrowia lipolytica IMUFRJ 50,682) (Costa, Andressa Maio da et al., 2020. “Poly(Ethylene Terephthalate) (PET) Degradation by Yarrowia Lipolytica: Investigations on Cell Growth, Enzyme Production and Monomers Consumption.” Process Biochemistry. https://doi.org/10.1016/j.procbio.2020.04.001; Chen, Zhuozhi et al., 2020. “Efficient Biodegradation of Highly Crystallized Polyethylene Terephthalate through Cell Surface Display of Bacterial PETase.” Science of the Total Environment. https://doi.org/10.1016/j.scitotenv.2019.136138), and microalga (Phaeodactylum tricornutum, Chlamydomonas reinhardtii CC-124 and CC-503) (Kim, Ji Won et al., 2020. “Functional Expression of Polyethylene Terephthalate-Degrading Enzyme (PETase) in Green Microalgae.” Microbial Cell Factories. https://doi.org/10.1186/s12934-020-01355-8; Moog, Daniel et al., 2019. “Using a Marine Microalga as a Chassis for Polyethylene Terephthalate (PET) Degradation.” Microbial Cell Factories. https://doi.org/10.1186/s12934-019-1220-z).

However, one major defect of IsPETase is that it is a heat-labile enzyme which exhibits a melting temperature (Tₘ) of only 48.8° C. It has been reported that IsPETase lost most of its enzyme activity after 24 hours of incubation at 30° C. (Son, Hyeoncheol Francis et al., 2019. “Rational Protein Engineering of Thermo-Stable PETase from Ideonella Sakaiensis for Highly Efficient PET Degradation.” ACS Catalysis 9 (4): 3519-26. https://doi.org/10.1021/acscatal.9b00568). Nonetheless, to maximize the degradation efficiency, it is more ideal that the PET-degrading enzyme applied in bioremediation can retain its enzymatic activity for a long period of time even under the harsh extracellular environment (e.g., conditions outside of the optimal conditions (temperature, pH, salt concentrations) of the wild-type PETase). Apparently, the low thermal stability of IsPETase will hamper its utilization for practical enzymatic degradation of PET. In order to improve the activity and stability of IsPETase, substantial research efforts have been made to determine the crystal structures of IsPETase and elucidate its catalytic mechanism (Joo, Seongjoon et al., 2018. “Structural Insight into Molecular Mechanism of Poly(Ethylene Terephthalate) Degradation.” Nature Communications. https://doi.org/10.1038/s41467-018-02881-1; Austin, Harry P. et al., 2018. “Characterization and Engineering of a Plastic-Degrading Aromatic Polyesterase.” Proceedings of the National Academy of Sciences of the United States of America. https://doi.org/10.1073/pnas. 1718804115), opening new opportunities for rational protein engineering of this enzyme. Employing the structural information on IsPETase, several studies have successfully introduced single point mutations that could afford 1.2- to 3.1-fold higher catalytic activity for PETdegradation (Taniguchi, Ikuo et al., 2019. “Biodegradation of PET: Current Status and Application Aspects.” ACS Catalysis 9 (5). https://doi.org/10.1021/acscatal.8b05171). Recently, Son et. al created an IsPETase^{S121E/D186H/R280A} variant that exhibits enhanced thermal stability (Tₘ=57.7° C.) and 14-fold higher PET degradation activity at 40° C. compared to wild type IsPETase (Son, Hyeoncheol Francis et al., 2019. “Rational Protein Engineering of Thermo-Stable PETase from Ideonella Sakaiensis for Highly Efficient PET Degradation.” ACS Catalysis 9 (4): 3519-26. https://doi.org/10.1021/acscatal.9b00568). More recently, Cui et al reported a computationally designed IsPETase^{L117F/Q119Y/T140D/W159H, G165A/I168R/A180I/S188Q/S214H/R280A} that possess a drastically improved thermal tolerance (Tₘ=79.8° C.) and an over two orders of magnitude enhancement in PET degradation activity (Cui, Ying-Lu et al., 2019. Computational Redesign of PETase for Plastic Biodegradation by GRAPE Strategy. https://doi.org/10.1101/787069). Lu et al. bioRxiv 2021.10.10.463845 (2021). doi: 10.1101/2021.10.10.463845 describes engineered PETases with improved activity.

### BRIEF SUMMARY OF THE INVENTION

In some embodiments, an engineered Leaf-branch compost cutinase (LCC) is provided comprising an amino acid sequence at least 90% (e.g., at least 95, 96, 97, 98, or 99%) identical to SEQ ID NO: 1 and having at least one mutation corresponding to a position relative to SEQ ID NO: 1 selected from the group consisting of D238K, T229Q, D129E and V233Q. In some embodiments, the engineered LCC comprises at least two or all of D238K, D129E and V233Q. In some embodiments, the engineered LCC further comprises mutation F243I, N246M, or both.

In some embodiments, the amino acid sequence is identical to SEQ ID NO:1 except has a mutation corresponding to T229Q.

In some embodiments, the engineered LCC comprises mutations corresponding to D129E, D238K, F243I and N246M. In some embodiments, the amino acid sequence is identical to SEQ ID NO:1 except comprises mutations corresponding to D129E, D238K, F243I and N246M.

In some embodiments, the amino acid sequence comprises SEQ ID NO:3 or SEQ ID NO: 4. In some embodiments, the amino acid sequence comprises any one of SEQ ID NOs: 5-16 or 21.

Also provided is an engineered Cut190 enzyme comprising an amino acid sequence at least 90% (e.g., at least 95, 96, 97, 98, or 99%) identical to SEQ ID NO:17 and having at least one mutation corresponding to a position relative to SEQ ID NO:17 of D250K. In some embodiments, the engineered Cut190 enzyme comprises SEQ ID NO:18.

Also provided is an engineered PHL7 enzyme comprising an amino acid sequence at least 90% (e.g., at least 95, 96, 97, 98, or 99%) identical to SEQ ID NO:19 and having at least one mutation corresponding to a position relative to SEQ ID NO:19 of R205K. In some embodiments, the engineered PHL7 enzyme comprises SEQ ID NO:20.

Also provided a polynucleotide encoding the engineered LCC, the engineered Cut190 enzyme or the engineered PHL7 enzyme as described above or elsewhere herein. In some embodiments, the engineered LCC, Cut190 enzyme, or PHL7 enzyme further comprises a signal peptide, e.g., for expression.

Also provided is a vector comprising a promoter operably linked to the polynucleotide as described above such that the promoter controls expression of the engineered LCC, Cut190 enzyme, or PHL7 enzyme.

Also provided is a host cell comprising the polynucleotide or the vector as described above. In some embodiments, the host cell is a microbial cell. In some embodiments, the host cell is a bacterial cell. In some embodiments, the bacterial cell is Pseudomonas putida. In some embodiments, the host cell is a fungal cell.

Also provided is a method of degrading poly(ethylene terephthalate) (PET) comprising contacting PET with the engineered LCC, Cut190 enzyme, or PHL7 enzyme as described above or elsewhere herein under conditions to degrade the PET. In some embodiments, the method comprises contacting the PET with a host cell that expresses and secretes the engineered LCC, Cut190 enzyme, or PHL7 enzyme. In some embodiments, the engineered LCC, Cut190 enzyme, or PHL7 enzyme is purified. In some embodiments, the conditions include an incubation at a temperature of between 25-100° C. In some embodiments, the temperature is 60-75° C. In some embodiments, the temperature is 70-85° C. or 70-100° C. or 90-100° or 95-100°. In some embodiments, the conditions include an incubation at a pH of 6-10 (e.g., 7-8.5, or 8).

### Definitions

A “Leaf-branch compost cutinase (LCC)” is from an esterase class of enzymes that catalyze the hydrolysis of polyethylene terephthalate (PET) plastic to monomeric mono-2-hydroxyethyl terephthalate (MHET) and terephthalic acid (TPA). See, e.g., Shirke, et al., Biochemistry 2018, 57, 7, 1190-1200. An example of a native LCC is SEQ ID NO:1. LCC activity can be determined by measuring the ability of an enzyme to degrade PET film, measured by the total amount of PET monomers released, or the change of weight of the film following incubation with the enzyme under set conditions for a set time period.

A “Cut190 enzyme” is a PET-degrading cutinase. Cut190 is described in, for example, Oda, Masayuki et al., Applied Microbiology and Biotechnology, 102 (23): 1-11, 2018 and provided in SEQ ID NO: 17.

A “PHL7 enzyme” is a PET-degrading cutinase. PHL7 is described in, for example, Sonnendecker, C., et al., ChemSusChem 15:9 (May 6, 2022), e202101062 and provided in SEG ID NO: 19.

A “polynucleotide” is a single- or double-stranded polymer of deoxyribonucleotide or ribonucleotide bases typically read from the 5′ to the 3′ end. Polynucleotides include RNA and DNA, and may be isolated from natural sources, synthesized in vitro, or prepared from a combination of natural and synthetic molecules. When the term is applied to double-stranded molecules it is used to denote overall length and will be understood to be equivalent to the term “base pairs”.

A “polypeptide” or “protein” is a polymer of amino acid residues joined by peptide bonds, whether produced naturally or synthetically.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, California, USA).

The phrase “substantial identity” or “substantially identical,” used in the context of two nucleic acids or polypeptides, refers to a sequence that has at least 70% sequence identity with a reference sequence. Alternatively, percent identity can be any integer from 70% to 100%. In some embodiments, a sequence is substantially identical to a reference sequence if the sequence has at least 70%, 75%, 80%, 85%, 90%, 91%, 92%, 93%, 94%, 95%, 96%, 97%, 98%, or 99% sequence identity to the reference sequence as determined using the methods described herein; preferably BLAST using standard parameters, as described below. Embodiments of the present disclosure provide for polypeptides that are substantially identical to any one or more of SEQ ID NO: 1-2 and that contain at least one amino acid substitution as described herein.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

Algorithms that are suitable for determining percent sequence identity and sequence similarity are the BLAST and BLAST 2.0 algorithms, which are described in Altschul et al. (1990) J. Mol. Biol. 215:403-410 and Altschul et al. (1977) Nucleic Acids Res. 25:3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10-5, and most preferably less than about 10-20.

An “amino acid mutation” refers to replacing the amino acid residue in a given position (e.g., the naturally occurring amino acid residue that occurs in a wild-type LCC (e.g., SEQ ID NO: 1) or a non-natural LCC (e.g., SEQ ID NO:2) or other enzyme (e.g., Cut190 or PHL7) with another amino acid residue (for example, other than the naturally-occurring residue). For example, the naturally occurring amino acid residue at position 238 of the wild-type LCC sequence (SEQ ID NO: 1) is aspartic acid (D) (D238); accordingly, an amino acid substitution at D238 refers to replacing the naturally occurring aspartic acid with any amino acid residue other than aspartic acid.

Individual substitutions to a polypeptide sequence that alters a single amino acid or a small percentage of amino acids in the encoded sequence results in a “conservatively modified variant” where the alteration results in the substitution of an amino acid with a chemically similar amino acid. The following amino acids are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

An amino acid residue “corresponding to an amino acid residue [X] in [specified sequence],” or an amino acid substitution “corresponding to an amino acid substitution [X] in [specified sequence]” and similar refers to an amino acid in a polypeptide of interest that aligns with the equivalent amino acid of a specified sequence when the polypeptide and the sequence are optimally aligned. For example, the amino acid corresponding to a position of SEQ ID NO:1 can be determined using an alignment algorithm such as BLAST. In some embodiments, “correspondence” of amino acid positions is determined by aligning SEQ ID NO: 1 to another LCC sequence. When a test LCC sequence differs from SEQ ID NO:1 (e.g., by changes in amino acids or addition or deletion of amino acids), it may be that a particular mutation associated with improved PET hydrolase activity will not be in the same numerical position number as it is in SEQ ID NO: 1, for example if the test LCC had an amino terminal signal sequence or one or more amino acid inserted relative to SEQ ID NO: 1 but the position will still “correspond” with the position in SEQ ID NO:1 as determined by alignment.

The term “host cell” refers to any cell capable of replicating and/or transcribing and/or translating a heterologous polynucleotide. Thus, a “host cell” refers to any prokaryotic cell (including but not limited to E. coli) or eukaryotic cell (including but not limited to yeast cells, mammalian cells, avian cells, amphibian cells, plant cells, fish cells, and insect cells), whether located in vitro or in vivo. For example, host cells may be located in a transgenic animal or transgenic plant. Host cells can be for example, transformed with heterologous polynucleotide.

A “vector” refers to a nucleic acid that includes a coding sequence and sequences necessary for expression of the coding sequence. The vector can be viral or non-viral. A “plasmid” is a non-viral vector, e.g., a nucleic acid molecule that encodes for genes and/or regulatory elements necessary for the expression of genes. A “viral vector” is a viral-derived nucleic acid that is capable of transporting another nucleic acid into a cell. A viral vector is capable of directing expression of a protein or proteins encoded by one or more genes carried by the vector when it is present in the appropriate environment. Examples for viral vectors include, but are not limited to retroviral, adenoviral, lentiviral and adeno-associated viral vectors.

### BRIEF DESCRIPTION OF THE DRAWINGS

FIG. 1: Thermostability of LCC and its variants. The melting temperature (Tₘ) of each enzyme was determined by Differential Scanning calorimetry. All measurement were conducted in triplicate (n=3).

FIG. 2: Comparison of PET-hydrolytic activity of LCC and its variants at reaction temperature of 60° C. (a) and 70° C. (b) against the semi-crystalized Goodfellow PET film. PET-hydrolytic activity of the tested enzymes was evaluated by measuring the amount of PET monomers (terephthalic acid (TPA) and Mono-(2-hydroxyethyl) terephthalate (MHET)) released after 24 hrs of reaction time. All measurement were conducted in triplicate (n=3).

FIG. 3: Comparison of PET-hydrolytic activity of LCC and its variants at reaction temperature of 60° C. (a) and 70° C. (b) against the amorphous Goodfellow PET film. PET-hydrolytic activity of the tested enzymes was evaluated by measuring the amount of PET monomers (terephthalic acid (TPA) and Mono-(2-hydroxyethyl) terephthalate (MHET)) released after 24 hrs of reaction time. All measurement were conducted in triplicate (n=3).

FIG. 4: Thermostability of LCC and ICCM variants incorporating the predicted mutations. Tm of each enzyme was determined by Differential Scanning calorimeters.

FIG. 5: PET-hydrolytic activity of LCC and ICCM variants incorporating the predicted mutations. PET-hydrolytic activity was evaluated by measuring the amount of PET monomers (the sum of TPA and MHET) released from hydrolyzing amorphous gf-PET with LCC and ICCM variants. The reaction temperature was at 70° C. KH2PO4-NaOH (pH 8) buffer was used for all enzymes shown in this figure.

FIG. 6: Thermostability of Cut190, PHL7 and their respective lysine variants. Tm of each enzyme was determined by Differential Scanning calorimeters.

FIG. 7: PET-hydrolytic activity of Cut190, PHL7 and their respective lysine variants. PET-hydrolytic activity was evaluated by measuring the amount of PET monomers (the sum of TPA and MHET) released from hydrolyzing amorphous gf-PET with LCC and ICCM variants. The reaction temperatures for each group of enzymes were 50° C. KH2PO4-NaOH (pH 8) buffer was used for all enzymes shown in this figure.

### DETAILED DESCRIPTION OF THE INVENTION

The present disclosure provides mutant forms of Leaf-branch compost cutinase (LCC) enzymes, Cut190 enzymes and PHL7 enzymes that exhibit enhanced enzymatic activity as compared to the corresponding wildtype enzyme. Mutant forms of LCC, Cut190 enzymes and PHIL7 enzymes described herein can also maintain catalytic activity across higher temperatures than a wild type enzyme.

The disclosure provides engineered (i.e., non-natural) LCC enzymes having certain amino acid changes compared to a control (e.g., native (SEQ ID NO:1)) LCC such that the engineered LCC has improved thermostability, improved PET hydrolytic activity at certain temperatures or pHs, or a combination thereof, compared to the control LCC. The control LCC will be a LCC lacking the recited mutations but otherwise identical, and typically will be the LCC sequence into which the mutations are introduced.

Exemplary mutants, which can be included for example in an amino acid sequence that is at least 90%, 91%, 92%, 93%, 94%, 95%, 96%, 97%, 98%, 99% or otherwise 100% identical to SEQ ID NO: 1 can include but are not limited to: D238K, D129E, T229Q, V233Q, D238K and D129E, D238K and V233Q, D129E and V233Q, or D238K, D129E, and V233Q. In addition to the mutations just listed, the engineered LCCs can further include, for example, F243I, N246M or both F243I and N246M.

Exemplary sequences include but are not limited to SEQ ID NOS: 3-4 as well as SEQ ID Nos: 5-16 and 21.

Also provided are engineered (i.e., non-natural) Cut190 enzymes having certain amino acid changes compared to a control (e.g., native (SEQ ID NO:17)) Cut190 enzyme such that the engineered Cut190 enzyme has improved thermostability, improved PET hydrolytic activity at certain temperatures or pHs, or a combination thereof, compared to the control Cut190 enzyme. The control Cut190 enzyme will be a Cut190 enzyme lacking the recited mutations but otherwise identical, and typically will be the Cut190 enzyme sequence into which the mutations are introduced. Exemplary Cut190 enzyme mutants, which can be included for example in an amino acid sequence that is at least 90%, 91%, 92%, 93%, 94%, 95%, 96%, 97%, 98%, 99% or otherwise 100% identical to SEQ ID NO:17 can include but are not limited to: D250K. Exemplary Cut190 enzymes containing the D25K mutation include those comprising SEQ ID NO:18.

Also provided are engineered (i.e., non-natural) PHL7 enzymes having certain amino acid changes compared to a control (e.g., native (SEQ ID NO:19)) PHL7 enzyme such that the engineered PHL7 enzyme has improved thermostability, improved PET hydrolytic activity at certain temperatures or pHs, or a combination thereof, compared to the control PHL7 enzyme. The control PHL7 enzyme will be a PHL7 enzyme lacking the recited mutations but otherwise identical, and typically will be the PHL7 enzyme sequence into which the mutations are introduced. Exemplary PHL7 enzyme mutants, which can be included for example in an amino acid sequence that is at least 90%, 91%, 92%, 93%, 94%, 95%, 96%, 97%, 98%, 99% or otherwise 100% identical to SEQ ID NO:19 can include but are not limited to: R205K. Exemplary PHL7 enzymes containing the R205K mutation include those comprising SEQ ID NO:20.

The disclosure also provides nucleic acids encoding the engineered LCCs described herein, e.g., engineered LCCs comprising an amino acid sequence substantially identical (e.g., at least 70, 80, 90, or 95%) identical to SEQ ID NO:1 and having at least one (e.g., 1, 2 or 3 or more) mutation corresponding to a position relative to SEQ ID NO: 1 selected from the group consisting of D238K, D129E, T229Q and V233Q. Also provided are nucleic acids encoding the Cut190 enzymes described herein, e.g., comprising an amino acid sequence that is at least 90%, 91%, 92%, 93%, 94%, 95%, 96%, 97%, 98%, 99% or otherwise 100% identical to SEQ ID NO:17 and including but are not limited to mutation D250K. Also provided are nucleic acids encoding the engineered PHL7 enzymes described above, e.g., comprising an amino acid sequence that is at least 90%, 91%, 92%, 93%, 94%, 95%, 96%, 97%, 98%, 99% or otherwise 100% identical to SEQ ID NO: 19 and including but are not limited to mutation: R205K. In some embodiments, the nucleic acids comprise a promoter operably linked to the coding sequence. The coding sequence can be codon optimized for the cell in which it will be expressed.

Nucleic acids encoding the polypeptides can be expressed using routine techniques in the field of recombinant genetics. Basic texts disclosing such techniques include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994-1999). Modifications of the polypeptides can additionally be made without diminishing biological activity. Some modifications may be made to facilitate the cloning, expression, or incorporation of a domain. The proteins described herein can be made using standard methods well known to those of skill in the art. Recombinant expression in a variety of host cells, including but not limited to prokaryotic cells such as E. coli, or other prokaryotic hosts are well known in the art.

Polynucleotides encoding the desired proteins in the complex, recombinant expression vectors, and host cells containing the recombinant expression vectors, as well as methods of making such vectors and host cells by recombinant methods are well known to those of skill in the art.

The polynucleotides may be synthesized or prepared by techniques well known in the art. Nucleotide sequences encoding the desired proteins may be synthesized, and/or cloned, and expressed according to techniques known to those of ordinary skill in the art. In some embodiments, the polynucleotide sequences will be codon optimized for a particular recipient using standard methodologies. For example, a DNA construct encoding a protein can be codon optimized for expression in microbial hosts, e.g., yeast or bacteria.

Examples of useful bacteria include, but are not limited to, Escherichia, Enterobacter, Azotobacter, Erwinia, Bacillus, Pseudomonas, Klebsiella, Proteus, Salmonella, Serratia, Shigella, Rhizobia, Vitreoscilla, and Paracoccus. The nucleic acid encoding the desired protein is operably linked to appropriate expression control sequences for each host. For E. coli this can include, for example, a promoter such as the T7, trp, or lambda promoters, a ribosome binding site and preferably a transcription termination signal. For P. putida this can include, for example, a promoter such as T7, lacI or similar promoter, a ribosome binding site, and preferably a secretion tag. The proteins may also be expressed in other cells, such as mammalian, insect, plant, or yeast cells.

In some embodiments, the polypeptide construct contains one or more affinity tags, e.g., for the purposes of detection or purification. A number of suitable tags can be included in the polypeptide constructs including, for example, those described by Kimple et al. (Curr Protoc Protein Sci. 2013; 73 (1): 9.9.1-9.9.23). Examples of affinity tags include, but are not limited to, a calmodulin binding peptide (CBP), a chitin binding domain (CBD), a dihyrofolate reductase (DHFR) moiety, a FLAG epitope, a glutathione S-transferase (GST) tag, a hemagglutinin (HA) tag; a maltose binding protein (MBP) moiety; a Myc epitope; a polyhistidine tag (e.g., HHHHHH); and streptavidin-binding peptides (e.g., those described in U.S. Pat. No. 5,506,121). An affinity tag may be included at one or more locations in the polypeptide construct. An affinity tag such as a streptavidin-binding peptide may reside, for example, at the N-terminus of the polypeptide construct or at the C-terminus of the polypeptide construct. In some embodiments, the linker peptide comprises an affinity tag, e.g., a FLAG epitope containing the sequence DYKDDDDK with or without additional amino acid residues.

The polypeptides described herein can be expressed intracellularly or can be secreted from the cell. In some embodiments a signal peptide is linked to the amino terminus of the expressed polypeptide such that the polypeptide is secreted from the cell.

Once expressed, the polypeptides can be purified according to standard procedures of the art, including ammonium sulfate precipitation, affinity columns, column chromatography, gel electrophoresis and the like (see, generally, R. Scopes, Protein Purification, Springer-Verlag, N.Y. (1982), Deutscher, Methods in Enzymology Vol. 182: Guide to Protein Purification., Academic Press, Inc. N.Y. (1990)). Substantially pure compositions of at least about 90 to 95% homogeneity (e.g., 98 to 99% or higher homogeneity) are provided in certain embodiments. Alternatively, in some embodiments, the polypeptide can be secreted from the cell and a crude, unpurified supernatant containing the polypeptide may be used.

Also provided are reaction mixtures comprising a plastic sample (e.g., a PET plastic) and one or more engineered LCC, Cut190 enzyme or PHL7 enzyme as described herein as well as methods of using such reaction mixtures to demonstrate the degradation of the PET plastics as measured by a percentage mass loss of the disc after incubation with the enzyme. The engineered LCC, Cut190 enzyme or PHL7 enzymes in the reaction mixtures can be in purified form or can be expressed in a host cell (i.e., the host cell expressing the enzyme can be in the reaction mixture). Some advantages of the described engineered LCC, Cut190 enzyme or PHL7 enzymes are that they can exhibit improved activity at elevated temperature while at the same time have the advantage of being able to degrade plastics at lower pH conditions and lower temperatures at a higher level of activity than previously described native or engineered LCC.

The time required for degrading a polyester containing material may vary depending on the polyester containing material itself (i.e., nature and origin of the plastic product, its composition, shape etc.), the precise enzyme and amount of enzyme used, as well as various process parameters (i.e., temperature, pH, additional agents, agitation etc.).

In some embodiments, the conditions of the degradation method include an ambient temperature, for example a temperature from 25-100° C. These temperatures can be especially useful in embodiments in which a host cell (e.g., a bacterial cell) expresses the engineered LCC, Cut190 enzyme or PHL7 enzyme and the host cell is contacted to the PET plastic in the reaction mixture. The precise temperature for optimal survival and enzyme expression by the cell can be selected.

Alternatively, in some embodiments, the engineered LCC, Cut190 enzyme or PHL7 enzyme is incubated with the PET plastic under higher temperatures, for example from 6000-75° C. or 70-85° C. or 70-100° C. or 90-100° C. or 95-100° C.

In some embodiments, the temperature is maintained below the glass transition temperature (Tg) of the PET plastic in the material being degraded. In some embodiments, the temperature is maintained at or above the glass transition temperature (Tg) of the PET plastic in the material being degraded. In some embodiments, the process is implemented in a continuous way, at a temperature at which the enzyme can be used several times and/or recycled.

A variety of pHs can be used with the described enzymes. In some embodiments, the engineered LCC, Cut190 enzyme or PHL7 enzyme and PET plastic are reacted under a pH of 6-10 (e.g., 6-8, 7-8.5, or 8-10). A more neutral pH range can be of use for example where cells expressing the enzyme are incubated with the plastic.

In some embodiments, the plastic containing material may be pretreated prior to be contacted with the engineered LCC, Cut190 enzyme or PHL7 enzyme, in order to physically change its structure, so as to increase the surface of contact between the plastic and the engineered LCC, Cut190 enzyme or PHL7 enzyme.

Optionally, monomers and/or oligomers resulting from the depolymerization may be recovered, sequentially or continuously. A single type of monomers and/or oligomers or several different types of monomers and/or oligomers may be recovered, depending on the starting plastic containing material.

In some embodiments, one or more engineered LCC, Cut190 enzyme or PHL7 enzyme as described herein is combined with a second enzyme (simultaneous or sequentially) to degrade a plastic product. For example in some embodiments, the second enzyme is a MHETase enzyme (see for example Palm et al., Nat Commun. 10:1717 (2019).

The recovered monomers and/or oligomers may be further purified, using all suitable purifying methods and conditioned in a re-polymerizable form. Examples of purifying methods include stripping process, separation by aqueous solution, steam selective condensation, filtration and concentration of the medium after the bioprocess, separation, distillation, vacuum evaporation, extraction, electrodialysis, adsorption, ion exchange, precipitation, crystallization, concentration and acid addition dehydration and precipitation, nanofiltration, acid catalyst treatment, semi continuous mode distillation or continuous mode distillation, solvent extraction, evaporative concentration, evaporative crystallization, liquid/liquid extraction, hydrogenation, azeotropic distillation process, adsorption, column chromatography, simple vacuum distillation and microfiltration, combined or not. Alternatively, the recovered/liberated monomers may be used by cells (either with or without explicit recovery) to be used as a carbon source for the production of a range products. This can be accomplished by co-incubation of the cells with the plastic and enzymes or in a sequential process.

The following examples exemplify aspects of the invention and are not intended to limited it.

### EXAMPLES

### Example 1

The Leaf-branch compost cutinase (LCC) and the Wildtype PETase (WT-PETase) were determined to have 47% identity and 63% similarity based on protein sequence alignment. FAST-PETase is a substantially improved mutant of WT-PETase with three key mutations (S121E, R224Q and N233K) that were predicted by Neural Network Analysis (Lu, H. et al. Deep learning redesign of PETase for practical PET degrading applications. bioRxiv 2021.10.10.463845 (2021). doi: 10.1101/2021.10.10.463845). Even though their native positions and amino acid identity were different than in PETase, mutations in LCC as listed below were predicted to improve LCC activity.

| | |
|---|---|
| S121E | D129E |
| R224Q | V233Q |
| N233K | D238K |

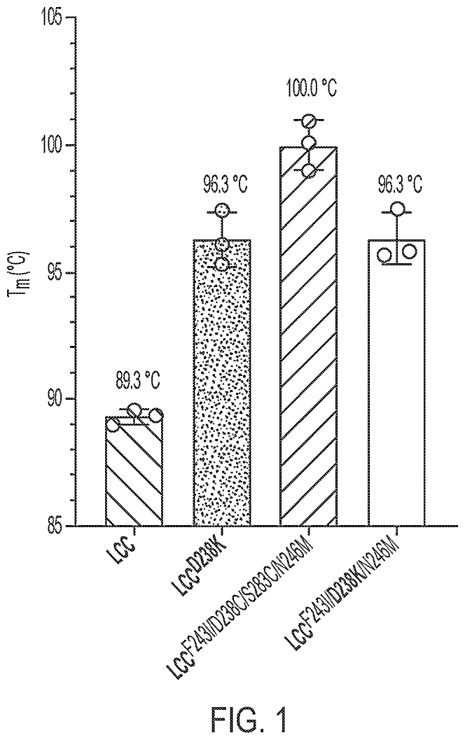

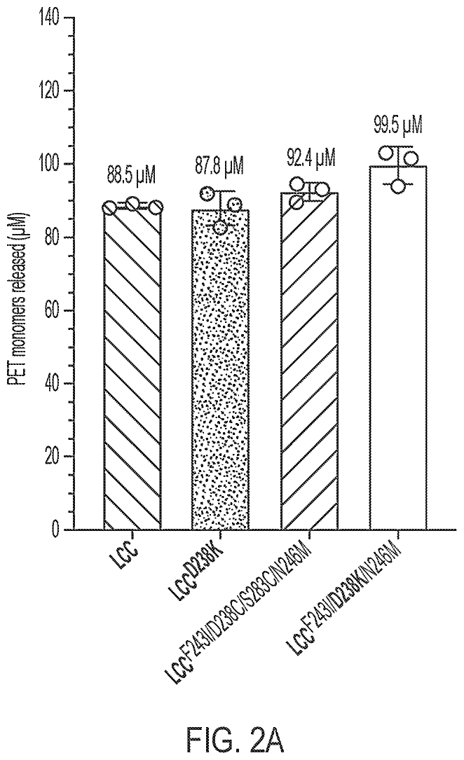

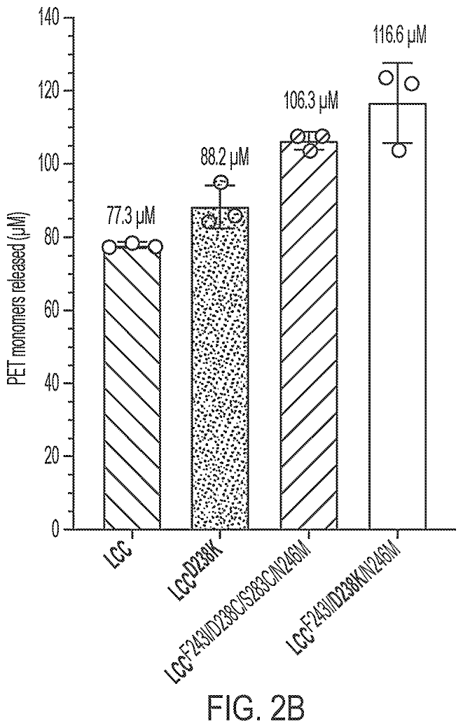

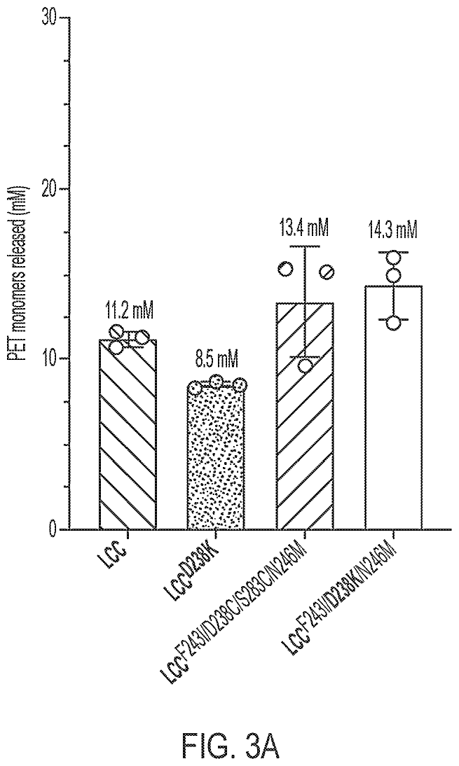

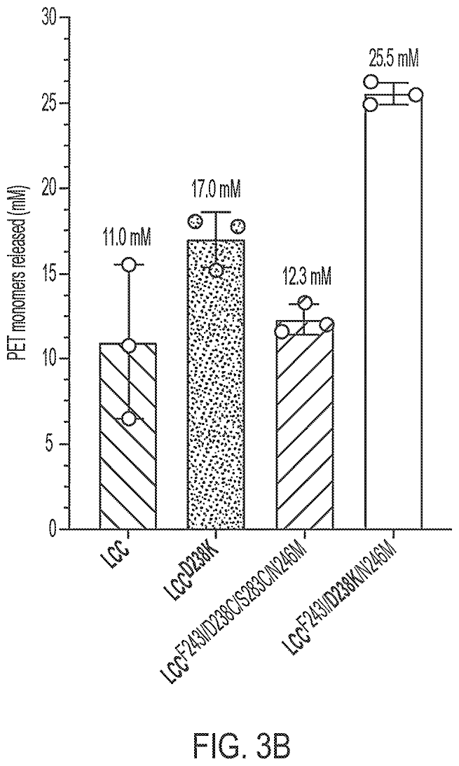

The introduction of mutation D238K to LCC (resulting in SEQ ID NO:3) resulted in substantially improved thermostability with ΔTₘ=7° C. (FIG. 1) and enhanced PET-hydrolytic activity (FIGS. 2 and 3) compared to the wildtype LCC.

LCC^{F243I/D238C/S283C/N246} (ICCM) (SEQ ID NO:2) is a LCC variant engineered by Tournier, V. et al. An engineered PET depolymerase to break down and recycle plastic bottles. Nature (2020). doi: 10.1038/s41586-020-2149-4. The thermostability of this variant was elevated by introducing a disulfide bond (D238C/S283C). However, this disulfide bond variant lost 28% of activity. We replaced the disulfide bond mutations with the mutation D238K for ICCM. The resultant mutant LCC^{F243I/D238K/N246M} (SEQ ID NO:4) exhibited an elevated ΔTₘ=7° C. relative to wildtype LCC, which is only 3.7° C. lower than the Tₘ of ICCM (FIG. 1). However, the PET-hydrolytic activity of LCC^{F243I/D238K/N246M} is significantly higher than ICCM (FIGS. 2 and 3), which indicates that the mutation D238K can confer improved thermostability to LCC and its variants and yet retain their enzymatic activity comparing to the disulfide bond mutants with a stability-activity trade-off.

### Example 2

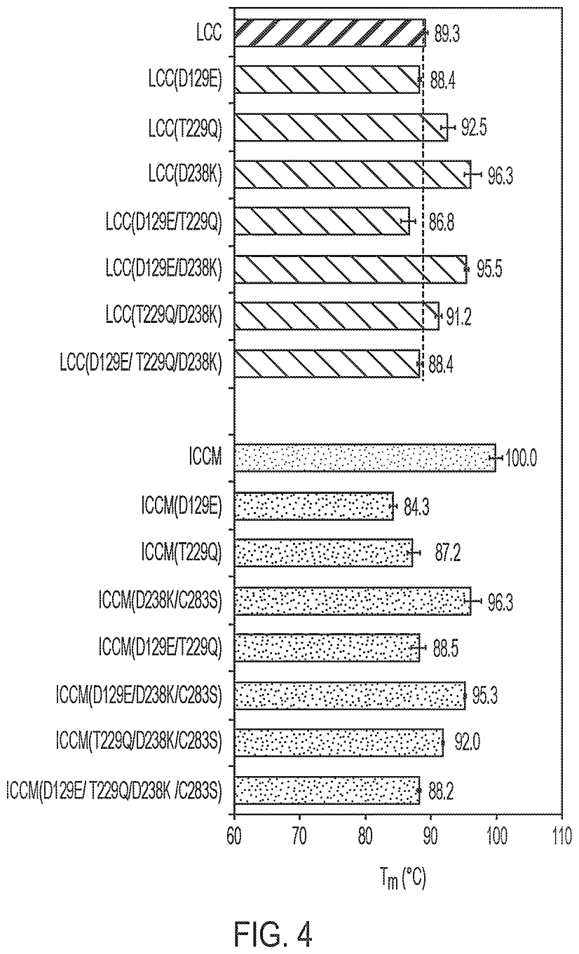

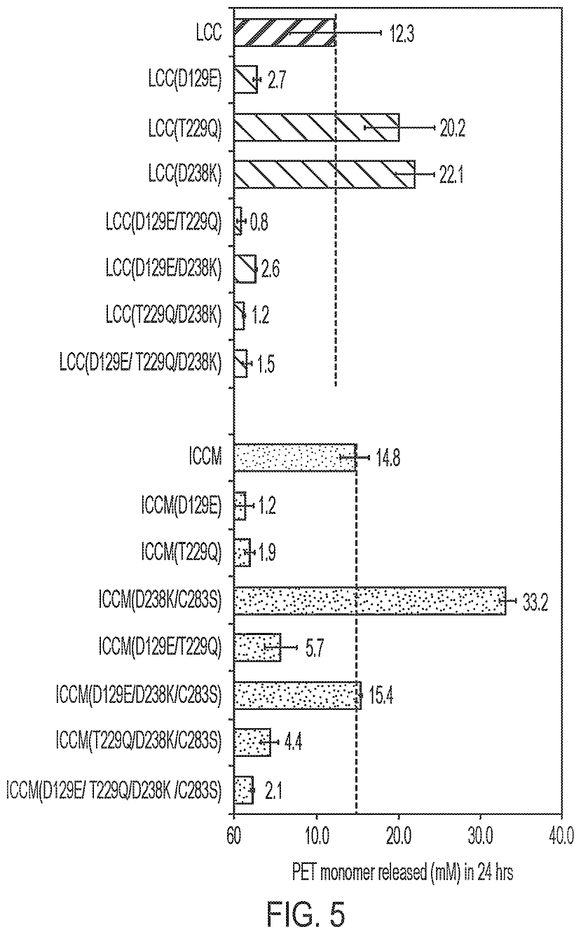

We assembled all 14 possible combinations (SEQ ID NO:3, 5-16 and 21) using D129E, T229Q and D238K mutations for both LCC and ICCM scaffolds. Thermostability analysis of the 14 mutants indicated that the LCC variants-LCC^{T229Q} (SEQ ID NO:6), LCC^{D238K} (SEQ ID NO:4), LCC^{D129E/D238K} (SEQ ID NO:10) and LCC^{T229Q/D238K} (SEQ ID NO:11) exhibited improved thermostability compared to the wildtype LCC (FIG. 4). The highest change in thermostability from LCC scaffold was observed for variants LCC^{D238K} with a Tm of 96.3° C. (ΔTₘ=7° C.). Regarding to the PET-hydrolytic activity, both LCC^{T229Q} and LCC^{D238K} exhibited higher PET-hydrolytic activity (FIG. 5) compared to the wildtype LCC.

In the previous study (Tournier, V. et al. An engineered PET depolymerase to break down and recycle plastic bottles. Nature (2020). doi: 10.1038/s41586-020-2149-4.), the thermostability of ICCM was elevated by introducing a disulfide bond (D238C/S283C). However, this disulfide bond variant lost 28% of activity.

Here, we replaced the disulfide bond mutations with the mutation D238K for ICCM and reverted the interacting cysteine back to its original serine. The resultant mutant ICCM^{D238K/C283S} (SEQ ID NO:21) exhibited an elevated ΔTₘ=7° C. relative to wildtype LCC, which is only 3.7° C. lower than the Tm of ICCM (FIG. 4). However, the PET-hydrolytic activity of ICCM^{D238K/C283S} is significantly higher than ICCM (FIG. 5), which indicates that the mutation D238K can confer improved thermostability to LCC and its variants and yet retain their enzymatic activity comparing to the disulfide bond mutants with a stability-activity trade-off.

Given the outstanding portability of the D238K to LCC and ICCM, we sought to further investigate the transferability of this lysine mutation to other PETase homologous enzymes. To this end, we introduced the corresponding lysine mutation to Cut190 (SEQ ID NO:17), (Kawai, F. et al. A novel Ca2+-activated, thermostabilized polyesterase capable of hydrolyzing polyethylene terephthalate from Saccharomonospora viridis AHK190. Appl. Microbiol. Biotechnol.98, 10053-10064 (2014)) and PHL7 (SEQ ID NO:19) (C. Sonnendecker, et al. Low Carbon Footprint Recycling of Post-Consumer PET Plastic with a Metagenomic Polyester Hydrolase. ChemSusChem 2022, 15, e202101062.)

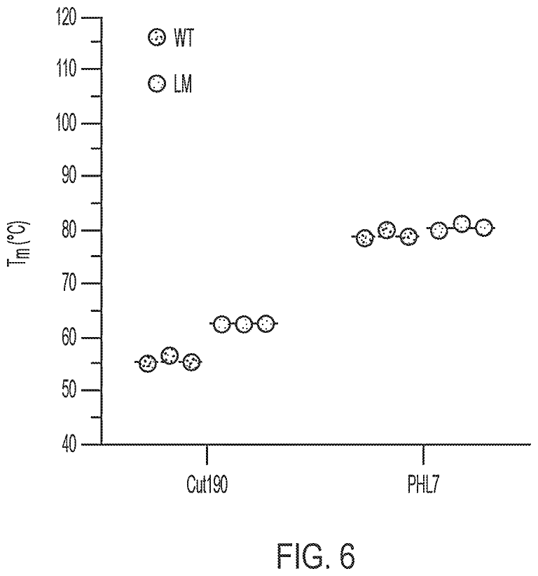

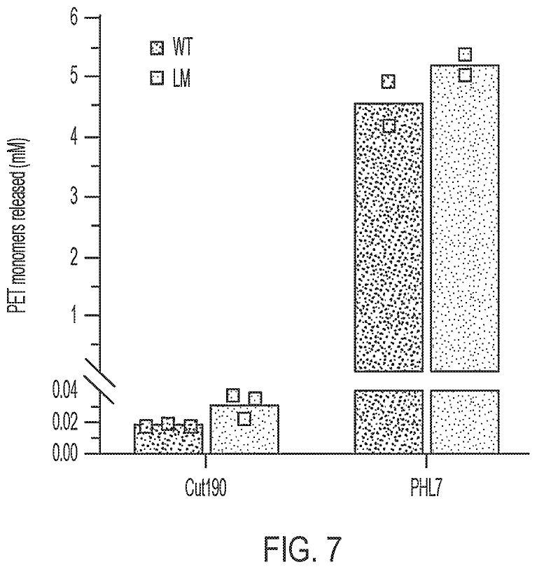

The resulting lysine mutation variant of Cut190 (Cut190D250K (SEQ ID NO:18)) showed substantially enhanced thermostability (ΔTm=7° C.) relative to its wildtype scaffold (FIG. 6). More importantly, the hydrolytic activity of both lysine mutation variants Cut190^{D250K} and PHIL7^{R205K} (SEQ ID NO:20) on amorphous gf-PET were notably higher than their respective scaffolds (FIG. 7), thus again showcasing the portability of the FAST-PETase lysine mutation.

SEQUENCES

| SEQ | | | |
|---|---|---|---|
| ID | | | |
| NO. | | | |
| 1 | 293 | Wild type Leaf-branch | MDGVLWRVRTAALMAALLALAAWALVWASPSVEAQSNPYQRGPN |
| | | compost cutinase (LCC) | PTRSALTADGPFSVATYTVSRLSVSGFGGGVIYYPTGTSLTFGGIAMSP |
| | | | GYTADASSLAWLGRRLASHGFVVLVINTNSRFDYPDSRASQLSAALNY |
| | | | LRTSSPSAVRARLDANRLAVAGHSMGGGGTLRIAEQNPSLKAAVPLT |
| | | | PWHTDKTFNTSVPVLIVGAEADTVAPVSQHAIPFYQNLPSTTPKVYVE |
| | | | LDNASHFAPNSNNAAISVYTISWMKLWVDNDTRYRQFLCNVNDPAL |
| | | | SDFRTNNRHCQ |
| 2 | 293 | ICCM (LCC^{F243I/D238C/S283C/N246M}) | MDGVLWRVRTAALMAALLALAAWALVWASPSVEAQSNPYQRGPN |
| | | | PTRSALTADGPFSVATYTVSRLSVSGFGGGVIYYPTGTSLTFGGIAMSP |
| | | | GYTADASSLAWLGRRLASHGFVVLVINTNSRFDYPDSRASQLSAALNY |
| | | | LRTSSPSAVRARLDANRLAVAGHSMGGGGTLRIAEQNPSLKAAVPLT |
| | | | PWHTDKTFNTSVPVLIVGAEADTVAPVSQHAIPFYQNLPSTTPKVYVE |
| | | | LCNASHIAPMSNNAAISVYTISWMKLWVDNDTRYRQFLCNVNDPAL |
| | | | CDFRTNNRHCQ |
| 3 | 293 | LCC^{D238K} | MDGVLWRVRTAALMAALLALAAWALVWASPSVEAQSNPYQRGPN |
| | | | PTRSALTADGPFSVATYTVSRLSVSGFGGGVIYYPTGTSLTFGGIAMSP |
| | | | GYTADASSLAWLGRRLASHGFVVLVINTNSRFDYPDSRASQLSAALNY |
| | | | LRTSSPSAVRARLDANRLAVAGHSMGGGGTLRIAEQNPSLKAAVPLT |
| | | | PWHTDKTFNTSVPVLIVGAEADTVAPVSQHAIPFYQNLPSTTPKVYVE |
| | | | LKNASHFAPNSNNAAISVYTISWMKLWVDNDTRYRQFLCNVNDPAL |
| | | | SDFRTNNRHCQ |
| 4 | 293 | LCC^{F243I/D238K/N246M} | MDGVLWRVRTAALMAALLALAAWALVWASPSVEAQSNPYQRGPN |
| | | | PTRSALTADGPFSVATYTVSRLSVSGFGGGVIYYPTGTSLTFGGIAMSP |
| | | | GYTADASSLAWLGRRLASHGFVVLVINTNSRFDYPDSRASQLSAALNY |
| | | | LRTSSPSAVRARLDANRLAVAGHSMGGGGTLRIAEQNPSLKAAVPLT |
| | | | PWHTDKTFNTSVPVLIVGAEADTVAPVSQHAIPFYQNLPSTTPKVYVE |
| | | | LKNASHIAPMSNNAAISVYTISWMKLWVDNDTRYRQFLCNVNDPAL |
| | | | SDFRTNNRHCQ |
| 5 | 259 | ICCM^{D129E} | MSNPYQRGPNPTRSALTADGPFSVATYTVSRLSVSGFGGGVIY |
| | | | YPTGTSLTFGGIAMSPGYTADASSLAWLGRRLASHGFVVLVINT |
| | | | NSRFDYPESRASQLSAALNYLRTSSPSAVRARLDANRLAVAGHS |
| | | | MGGGGTLRIAEQNPSLKAAVPLTPWHTDKTFNTSVPVLIVGAE |
| | | | ADTVAPVSQHAIPFYQNLPSTTPKVYVELCNASHIAPMSNNAAI |
| | | | SVYTISWMKLWVDNDTRYRQFLCNVNDPALCDFRTNNRHCQ |
| 6 | 259 | LCC^{T229Q} | MSNPYQRGPNPTRSALTADGPFSVATYTVSRLSVSGFGGGVIY |
| | | | YPTGTSLTFGGIAMSPGYTADASSLAWLGRRLASHGFVVLVINT |
| | | | NSRFDYPDSRASQLSAALNYLRTSSPSAVRARLDANRLAVAGHS |
| | | | MGGGGTLRIAEQNPSLKAAVPLTPWHTDKTFNTSVPVLIVGAE |
| | | | ADTVAPVSQHAIPFYQNLPSQTPKVYVELDNASHFAPNSNNAA |
| | | | ISVYTISWMKLWVDNDTRYRQFLCNVNDPALSDFRTNNRHCQ |
| 7 | 259 | ICCM^{T229Q} | MSNPYQRGPNPTRSALTADGPFSVATYTVSRLSVSGFGGGVIY |
| | | | YPTGTSLTFGGIAMSPGYTADASSLAWLGRRLASHGFVVLVINT |
| | | | NSRFDYPDSRASQLSAALNYLRTSSPSAVRARLDANRLAVAGHS |
| | | | MGGGGTLRIAEQNPSLKAAVPLTPWHTDKTFNTSVPVLIVGAE |
| | | | ADTVAPVSQHAIPFYQNLPSQTPKVYVELCNASHIAPMSNNAA |
| | | | ISVYTISWMKLWVDNDTRYRQFLCNVNDPALCDFRTNNRHCQ |
| 8 | 259 | LCC^{D129E} | MSNPYQRGPNPTRSALTADGPFSVATYTVSRLSVSGFGGGVIY |
| | | | YPTGTSLTFGGIAMSPGYTADASSLAWLGRRLASHGFVVLVINT |
| | | | NSRFDYPESRASQLSAALNYLRTSSPSAVRARLDANRLAVAGHS |
| | | | MGGGGTLRIAEQNPSLKAAVPLTPWHTDKTFNTSVPVLIVGAE |
| | | | ADTVAPVSQHAIPFYQNLPSTTPKVYVELDNASHFAPNSNNAAI |
| | | | SVYTISWMKLWVDNDTRYRQFLCNVNDPALSDFRTNNRHCQ |
| 9 | 259 | LCC^{D129E/T229Q} | MSNPYQRGPNPTRSALTADGPFSVATYTVSRLSVSGFGGGVIY |
| | | | YPTGTSLTFGGIAMSPGYTADASSLAWLGRRLASHGFVVLVINT |
| | | | NSRFDYPESRASQLSAALNYLRTSSPSAVRARLDANRLAVAGHS |
| | | | MGGGGTLRIAEQNPSLKAAVPLTPWHTDKTFNTSVPVLIVGAE |
| | | | ADTVAPVSQHAIPFYQNLPSQTPKVYVELDNASHFAPNSNNAA |
| | | | ISVYTISWMKLWVDNDTRYRQFLCNVNDPALSDFRTNNRHCQ |
| 10 | 259 | LCC^{D129E/D238K} | MSNPYQRGPNPTRSALTADGPFSVATYTVSRLSVSGFGGGVIY |
| | | | YPTGTSLTFGGIAMSPGYTADASSLAWLGRRLASHGFVVLVINT |
| | | | NSRFDYPESRASQLSAALNYLRTSSPSAVRARLDANRLAVAGHS |
| | | | MGGGGTLRIAEQNPSLKAAVPLTPWHTDKTFNTSVPVLIVGAE |
| | | | ADTVAPVSQHAIPFYQNLPSTTPKVYVELKNASHFAPNSNNAAI |
| | | | SVYTISWMKLWVDNDTRYRQFLCNVNDPALSDFRTNNRHCQ |
| 11 | 259 | LCC^{T229Q/D238K} | MSNPYQRGPNPTRSALTADGPFSVATYTVSRLSVSGFGGGVIY |
| | | | YPTGTSLTFGGIAMSPGYTADASSLAWLGRRLASHGFVVLVINT |
| | | | NSRFDYPDSRASQLSAALNYLRTSSPSAVRARLDANRLAVAGHS |
| | | | MGGGGTLRIAEQNPSLKAAVPLTPWHTDKTFNTSVPVLIVGAE |
| | | | ADTVAPVSQHAIPFYQNLPSQTPKVYVELKNASHFAPNSNNAA |
| | | | ISVYTISWMKLWVDNDTRYRQFLCNVNDPALSDFRTNNRHCQ |
| 12 | 259 | ICCM^{D129E/T229Q} | MSNPYQRGPNPTRSALTADGPFSVATYTVSRLSVSGFGGGVIY |
| | | | YPTGTSLTFGGIAMSPGYTADASSLAWLGRRLASHGFVVLVINT |
| | | | NSRFDYPESRASQLSAALNYLRTSSPSAVRARLDANRLAVAGHS |
| | | | MGGGGTLRIAEQNPSLKAAVPLTPWHTDKTFNTSVPVLIVGAE |
| | | | ADTVAPVSQHAIPFYQNLPSQTPKVYVELCNASHIAPMSNNAA |
| | | | ISVYTISWMKLWVDNDTRYRQFLCNVNDPALCDFRTNNRHCQ |
| 13 | 259 | ICCM^{D129E/D238K/C283S} | MSNPYQRGPNPTRSALTADGPFSVATYTVSRLSVSGFGGGVIY |
| | | | YPTGTSLTFGGIAMSPGYTADASSLAWLGRRLASHGFVVLVINT |
| | | | NSRFDYPESRASQLSAALNYLRTSSPSAVRARLDANRLAVAGHS |
| | | | MGGGGTLRIAEQNPSLKAAVPLTPWHTDKTFNTSVPVLIVGAE |
| | | | ADTVAPVSQHAIPFYQNLPSTTPKVYVELKNASHIAPMSNNAAI |
| | | | SVYTISWMKLWVDNDTRYRQFLCNVNDPALSDFRTNNRHCQ |
| 14 | 259 | ICCM^{T229Q/D238K/C283S} | MSNPYQRGPNPTRSALTADGPFSVATYTVSRLSVSGFGGGVIY |
| | | | YPTGTSLTFGGIAMSPGYTADASSLAWLGRRLASHGFVVLVINT |
| | | | NSRFDYPDSRASQLSAALNYLRTSSPSAVRARLDANRLAVAGHS |
| | | | MGGGGTLRIAEQNPSLKAAVPLTPWHTDKTFNTSVPVLIVGAE |
| | | | ADTVAPVSQHAIPFYQNLPSQTPKVYVELKNASHIAPMSNNAA |
| | | | ISVYTISWMKLWVDNDTRYRQFLCNVNDPALSDFRTNNRHCQ |
| 15 | 259 | ICCM^{D129E/T229Q/D238K /C283S} | MSNPYQRGPNPTRSALTADGPFSVATYTVSRLSVSGFGGGVIY |
| | | | YPTGTSLTFGGIAMSPGYTADASSLAWLGRRLASHGFVVLVINT |
| | | | NSRFDYPESRASQLSAALNYLRTSSPSAVRARLDANRLAVAGHS |
| | | | MGGGGTLRIAEQNPSLKAAVPLTPWHTDKTFNTSVPVLIVGAE |
| | | | ADTVAPVSQHAIPFYQNLPSQTPKVYVELKNASHIAPMSNNAA |
| | | | ISVYTISWMKLWVDNDTRYRQFLCNVNDPALSDFRTNNRHCQ |
| 16 | 259 | LCC^{D129E/T229Q/D238K} | MSNPYQRGPNPTRSALTADGPFSVATYTVSRLSVSGFGGGVIY |
| | | | YPTGTSLTFGGIAMSPGYTADASSLAWLGRRLASHGFVVLVINT |
| | | | NSRFDYPESRASQLSAALNYLRTSSPSAVRARLDANRLAVAGHS |
| | | | MGGGGTLRIAEQNPSLKAAVPLTPWHTDKTFNTSVPVLIVGAE |
| | | | ADTVAPVSQHAIPFYQNLPSQTPKVYVELKNASHFAPNSNNAA |
| | | | ISVYTISWMKLWVDNDTRYRQFLCNVNDPALSDFRTNNRHCQ |
| 17 | 308 | Cut190 | MRIRRQAGTGARASMARAIGVMTTALAVLVGAVGGVAGAEV |
| | | | STAQDNPYERGPDPTEDSIEAIRGPFSVATERVSSFASGFGGGTI |
| | | | YYPRETDEGTFGAVAVAPGFTASQGSMSWYGERVASQGFIVF |
| | | | TIDTNTRLDQPGQRGRQLLAALDYLVERSDRKVRERLDPNRLA |
| | | | VMGHSMGGGGSLEATVMRPSLKASIPLTPWNLDKTWGQVQ |
| | | | VPTFIIGAELDTIASVRTHAKPFYESLPSSLPKAYMELDGATHFAP |
| | | | NIPNTTIAKYVISWLKRFVDEDTRYSQFLCPNPTDRAIEEYRSTCP |
| | | | YLEHH |
| 18 | 308 | Cut190^{D250K} | MRIRRQAGTGARASMARAIGVMTTALAVLVGAVGGVAGAEV |
| | | | STAQDNPYERGPDPTEDSIEAIRGPFSVATERVSSFASGFGGGTI |
| | | | YYPRETDEGTFGAVAVAPGFTASQGSMSWYGERVASQGFIVF |
| | | | TIDTNTRLDQPGQRGRQLLAALDYLVERSDRKVRERLDPNRLA |
| | | | VMGHSMGGGGSLEATVMRPSLKASIPLTPWNLDKTWGQVQ |
| | | | VPTFIIGAELDTIASVRTHAKPFYESLPSSLPKAYMELKGATHFAP |
| | | | NIPNTTIAKYVISWLKRFVDEDTRYSQFLCPNPTDRAIEEYRSTCP |
| | | | YLEHH |
| 19 | 259 | PHL7 | MANPYERGPDPTESSIEAVRGPFAVAQTTVSRLQADGFGGGTI |
| | | | YYPTDTSQGTFGAVAISPGFTAGQESIAWLGPRIASQGFVVITID |
| | | | TITRLDQPDSRGRQLQAALDHLRTNSVVRNRIDPNRMAVMGH |
| | | | SMGGGGALSAAANNTSLEAAIPLQGWHTRKNWSSVRTPTLVV |
| | | | GAQLDTIAPVSSHSEAFYNSLPSDLDKAYMELRGASHLVSNTPD |
| | | | TTTAKYSIAWLKRFVDDDLRYEQFLCPAPDDFAISEYRSTCPF |
| 20 | 259 | PHL7^{R205K} | MANPYERGPDPTESSIEAVRGPFAVAQTTVSRLQADGFGGGTI |
| | | | YYPTDTSQGTFGAVAISPGFTAGQESIAWLGPRIASQGFVVITID |
| | | | TITRLDQPDSRGRQLQAALDHLRTNSVVRNRIDPNRMAVMGH |
| | | | SMGGGGALSAAANNTSLEAAIPLQGWHTRKNWSSVRTPTLVV |
| | | | GAQLDTIAPVSSHSEAFYNSLPSDLDKAYMELKGASHLVSNTPD |
| | | | TTTAKYSIAWLKRFVDDDLRYEQFLCPAPDDFAISEYRSTCPF |
| 21 | 259 | ICCM^{D238K/C283S} | MSNPYQRGPNPTRSALTADGPFSVATYTVSRLSVSGFGGGVIY |
| | | | YPTGTSLTFGGIAMSPGYTADASSLAWLGRRLASHGFVVLVINT |
| | | | NSRFDYPDSRASQLSAALNYLRTSSPSAVRARLDANRLAVAGHS |
| | | | MGGGGTLRIAEQNPSLKAAVPLTPWHTDKTFNTSVPVLIVGAE |
| | | | ADTVAPVSQHAIPFYQNLPSTTPKVYVELKNASHIAPMSNNAAI |
| | | | SVYTISWMKLWVDNDTRYRQFLCNVNDPALSDFRTNNRHCQ |

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. An engineered Leaf-branch compost cutinase (LCC) comprising an amino acid sequence at least 90% identical to SEQ ID NO:1 and having at least one mutation corresponding to a position relative to SEQ ID NO: 1 selected from the group consisting of D238K, T229Q, D129E and V233Q.

2. The engineered LCC of claim 1, comprising at least two or all of D238K, D129E and V233Q.

3. The engineered LCC of claim 1, further comprising mutation F243I, N246M, or both.

4. The engineered LCC of claim 1, wherein the amino acid sequence is identical to SEQ ID NO:1 except has a mutation corresponding to T229Q.

5. The engineered LCC of claim 1, comprising mutations corresponding to D129E, D238K, F243I and N246M.

6. The engineered LCC of claim 5, wherein the amino acid sequence is identical to SEQ ID NO: 1 except comprises mutations corresponding to D129E, D238K, F243I and N246M.

7. The engineered LCC of claim 1, wherein the amino acid sequence comprises SEQ ID NO: 3 or SEQ ID NO:4.

8. The engineered LCC of claim 1, wherein the amino acid sequence comprises any one of SEQ ID NOs: 5-16 or 21.

9. An engineered Cut190 enzyme comprising an amino acid sequence at least 90% identical to SEQ ID NO:17 and having at least one mutation corresponding to a position relative to SEQ ID NO:17 of D250K.

10. The engineered Cut190 enzyme of claim 9, comprising SEQ ID NO:18.

11. An engineered PHL7 enzyme comprising an amino acid sequence at least 90% identical to SEQ ID NO:19 and having at least one mutation corresponding to a position relative to SEQ ID NO: 19 of R205K.

12. The engineered PHL7 enzyme of claim 11, comprising SEQ ID NO:20.

13. A polynucleotide encoding the engineered LCC of claim 1.

14. (canceled)

15. A vector comprising a promoter operably linked to the polynucleotide of claim 13.

16. A host cell comprising the polynucleotide of claim 13.

17. The host cell of claim 16, wherein the host cell is a microbial cell.

18. The host cell of claim 16, wherein the host cell is a bacterial cell.

19. The host cell of claim 18, wherein the bacterial cell is Pseudomonas putida.

20. The host cell of claim 16, wherein the host cell is a fungal cell.

21. A method of degrading poly(ethylene terephthalate) (PET) comprising contacting PET with the engineered LCC of claim 1 under conditions to degrade the PET.

22-27. (canceled)
